(19) United States
(12) Patent Application Publication
(10) Pub. No.: US 2023/0166029 A1
(43) Pub. Date: Jun. 1, 2023

(54) SHIFT-HEAD INSERTER DEVICE WITH AUTOMATIC INSERTION AND RETRACTION

(71) Applicant: Eli Lilly and Company, Indianapolis, IN (US)

(72) Inventors: Mary Teresa CARTER, Boston, MA (US); Louise Emma VAN DEN HEUVEL, Medford, MA (US)

(21) Appl. No.: 17/998,943

(22) PCT Filed: May 26, 2021
(86) PCT No.: PCT/US2021/034181

§ 371 Date: Nov. 16, 2022

Related U.S. Application Data
(60) Provisional application No. 63/031,044, filed on May 28, 2020.

(51) Int. Cl. A61M 5/158

(52) U.S. Cl. CPC A61M 5/158; A61M 2005/1585

(57) ABSTRACT

An inserter device is disclosed with an automatic insertion mechanism that shifts an infusion head and a needle assembly from a stowed configuration to an insertion configuration in which a cannula of the infusion head and an introducer needle of the needle assembly are automatically inserted into a patients skin. The inserter device also includes an automatic retraction mechanism that shifts the needle assembly to a retracted configuration in which the introducer needle is removed from the patients skin.

### FIELD OF THE DISCLOSURE

The present disclosure relates to drug infusion systems, more specifically an inserter device that can be used to insert a cannula into the subcutaneous space of a patient, and a method of using the same.

### BACKGROUND OF THE DISCLOSURE

Infusion sets are used to deliver a drug to the subcutaneous space of a patient. The head assembly of the infusion set has a fluid path in the form of a stainless-steel needle or soft cannula that must be inserted to the correct depth in the subcutaneous tissue. To insert a soft cannula, a stainless-steel introducer needle is used. The introducer needle opens a hole in the tissue to allow the cannula to enter and provides stiffness for the cannula as it is inserted. After insertion, the introducer needle is removed.

The insertion and retraction of the introducer needle constitute separate steps which may be performed manually by the patient or automated through an inserter. Most commercially-available inserters automate the insertion of the introducer needle. After insertion, the introducer needle is typically retracted manually.

Another important consideration is which components move during insertion. Most inserters are “shift head” devices 1000, in which the fluid path 1602 is irreversibly coupled to the infusion head 1601 and travels with the infusion head 1601 during the insertion process as an integrated infusion head assembly 1600 (FIG. 1). In contrast, other inserters are “shift fluid-path” devices 2000, in which the fluid path 2602 starts off separated from the infusion head 2601 and is advanced toward and joined to the infusion head 2601 during the insertion process (FIG. 2).

“Shift head” devices 1000 (FIG. 1) in which the fluid path 1602 is an integral part of the infusion head assembly 1600 are mechanically robust because the entire infusion head assembly 1600 moves as a single part, no snaps or latches are required. Shifting the entire infusion head assembly 1600 also means that there are no gaps between components that might allow water ingress between them. Eliminating water ingress reduces infection risk. In contrast, for “shift fluid-path” devices 2000 (FIG. 2), the infusion head 2601 and the adhesive patch are initially applied to the skin when the patient places the device 2000 on his or her skin. When the patient or the patient's caregiver activates the device 2000, the fluid path 2602 is shifted towards the skin and latched into the infusion head 2601. Because the large adhesive patch and infusion head 2601 start off attached to the skin, “shift fluid-path” devices 2000 can be smaller and more compact. However, they have additional mechanical complexity due to the increased number of components required, and the dynamic interface. The latching of the fluid path 2602 into the infusion head 2601 must also be controlled to minimize unwanted crevices near the insertion site. The clips that capture the fluid path 2602 must also have some tolerance to them, so that—even if it is captured correctly—the fluid path 2602 may wobble slightly within the clips, potentially causing discomfort. Taken as a whole, “shift head” devices 1000 present several advantages over “shift fluid-path” devices 2000.

To minimize patient burden, reduce user-error, and provide as painless an insertion experience as possible, it is desirable to have an inserter device that provides both auto-insertion and auto-retraction of the introducer needle. Moreover, to ensure mechanical robustness, it is desirable to have an inserter device that shifts the entire infusion head assembly, rather than the fluid path. It is therefore an object of this invention to provide an inserter device that provides both auto-insertion and auto-retraction of the introducer needle, while also shifting the entire infusion head assembly.

### SUMMARY

The present disclosure relates to an inserter device having an automatic insertion mechanism that shifts an infusion head and a needle assembly from a stowed configuration to an insertion configuration in which a cannula of the infusion head and an introducer needle of the needle assembly are inserted into a patient's skin. The inserter device also includes an automatic retraction mechanism that automatically shifts the needle assembly to a retracted configuration in which the introducer needle is removed from the patient's skin.

According to an exemplary embodiment of the present disclosure, an inserter device is disclosed including a housing, an actuator, an infusion head including a cannula, a push plate removably coupled to the infusion head, a needle assembly including an introducer needle, an insertion mechanism configured to automatically move the infusion head, the push plate, and the needle assembly from a stowed configuration in which the cannula and the introducer needle are positioned within the housing to an insertion configuration in which the cannula and the introducer needle extend from the housing in response to movement of the actuator, and a retraction mechanism configured to automatically move the needle assembly from the insertion configuration to a retracted configuration in which the introducer needle is positioned within the housing.

According to another exemplary embodiment of the present disclosure, an inserter device is disclosed including a housing, an actuator, an infusion head including a cannula, a push plate removably coupled to the infusion head, a needle assembly including an introducer needle, an automatic insertion mechanism, and a shift plate having a stowed configuration in which the automatic insertion mechanism is loaded against the shift plate with the infusion head and the needle assembly positioned within the housing, an insertion configuration in which the automatic insertion mechanism moves the shift plate, the infusion head, the push plate, and the needle assembly relative to the housing, the cannula and the introducer needle extending from the housing, and an intermediate configuration in which the automatic insertion mechanism moves the shift plate relative to the infusion head, the push plate, and the needle assembly.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a schematic view of a prior art example of an inserter device that shifts the infusion set head;

FIG. 2 is a schematic view of a prior art example of an inserter device that shifts the cannula;

FIG. 3 is an exploded view of an embodiment of the present inserter;

FIG. 4 is a perspective view of a lower housing of the inserter device of FIG. 3;

FIG. 5 is a perspective view of an upper housing of the inserter device of FIG. 3;

FIG. 6 is a perspective view of a carriage of the inserter device of FIG. 3;

FIG. 7 is a perspective view of a needle assembly of the inserter device of FIG. 3;

FIG. 8 is a perspective view of a shift plate of the inserter device of FIG. 3;

FIG. 9 is a perspective view of a push plate of the inserter device of FIG. 3;

FIG. 10 is a perspective view of an infusion head assembly of the inserter device of FIG. 3;

FIG. 11 is a perspective view of a safety assembly of the inserter device of FIG. 3;

FIG. 12 is a cross-sectional view of the inserter device of FIG. 3 in a stowed configuration with the safety assembly in a locked configuration, where the cross-section is taken along direction L;

FIG. 13 is a partial perspective view of the inserter device of FIG. 3 in the stowed configuration with the safety assembly in the locked configuration;

FIG. 14 is a partial perspective view of the inserter device of FIG. 3 in a triggered configuration with the safety assembly in an unlocked configuration;

FIG. 15 is a cross-sectional view of the inserter device of FIG. 3 in the triggered configuration just before needle insertion, where the cross-section is parallel to direction L;

FIG. 16 is a cross-sectional view of the inserter device of FIG. 3 in the same triggered configuration as is seen in FIG. 15, where the cross-section is taken along direction W;

FIG. 17 is a cross-sectional view of the inserter device of FIG. 3 in an inserted configuration after needle insertion but before retraction, where the cross-section is parallel to direction L;

FIG. 18 is a cross-sectional view of the inserter device of FIG. 3 in the same inserted configuration as is seen in FIG. 17, where the cross-section is taken along direction W;

FIG. 19 is a partial cross-sectional perspective of the inserter device of FIG. 3 in the inserted configuration showing a headlock in a locked position;

FIG. 20 is a partial cross-sectional perspective of the inserter device of FIG. 3 in an intermediate configuration showing the headlock in an unlocked position;

FIG. 21 is a cross-sectional view of the inserter device of FIG. 3 in the intermediate configuration just before retraction, where the cross-section is parallel to direction L;

FIG. 22 is a cross-sectional view of the inserter device of FIG. 3 in the same intermediate configuration as is seen in FIG. 21, where the cross-section is taken along direction W;

FIG. 23 is a cross-sectional view of the inserter device of FIG. 3 in a retracted configuration, where the cross-section is parallel to direction L;

FIG. 24 is a cross-sectional of the inserter device of FIG. 3 in the same retracted configuration as is seen in FIG. 23, where the cross-section is taken along direction W;

FIG. 25 is a top perspective view of the inserter device of FIG. 3 with the upper housing removed;

FIG. 26 is a cross-sectional view of the inserter device of FIG. 3 showing housing guides, where the cross-section is taken along direction L;

FIG. 27 is a cross-sectional view of the inserter device of FIG. 3 showing the housing guides, where the cross-section is taken parallel to direction W;

FIG. 28 is an exploded view of an embodiment of the present inserter device that includes a holder;

FIG. 29 is a cross-sectional view of the inserter device of FIG. 28, where the cross-section is taken along direction L;

FIG. 30 is a partial cross-sectional view of the inserter device of FIG. 28 showing a carriage, a clip holder, and a holder in a locked configuration; and

FIG. 31 is a partial cross-sectional view of the inserter device of FIG. 28 showing the carriage, clip holder, and holder in an unlocked configuration.

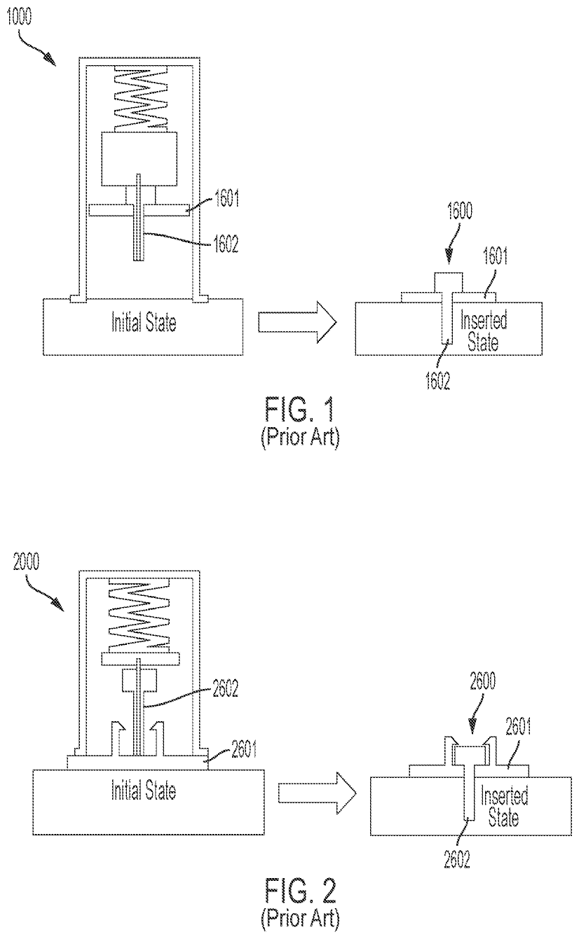

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

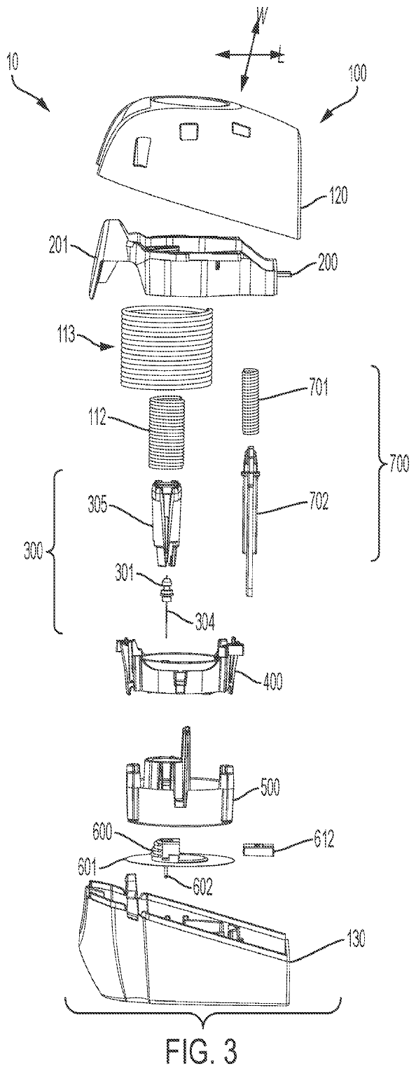

An exemplary inserter device 10 of the present disclosure is shown in FIG. 3. The inserter device 10 has a length measured along axis L and a width taken along axis W. The inserter device 10 of FIG. 3 includes a housing 100 including an upper housing 120 and a lower housing 130, a carriage 200 doubling as an actuator in the form of a button 201, a needle assembly 300, a shift plate 400, a push plate 500, an infusion head assembly 600, and a safety assembly 700. In use, the inserter device 10 provides both automatic insertion and automatic retraction of the needle assembly 300. Inserter device 10 automatically inserts and retracts the needle assembly 300 by shifting the infusion head assembly 600 to insert a cannula 602 into a patient's skin. The cannula 602 is then used for delivering a drug (not shown) into the patient's skin. Each element of the inserter device 10 is described further below.

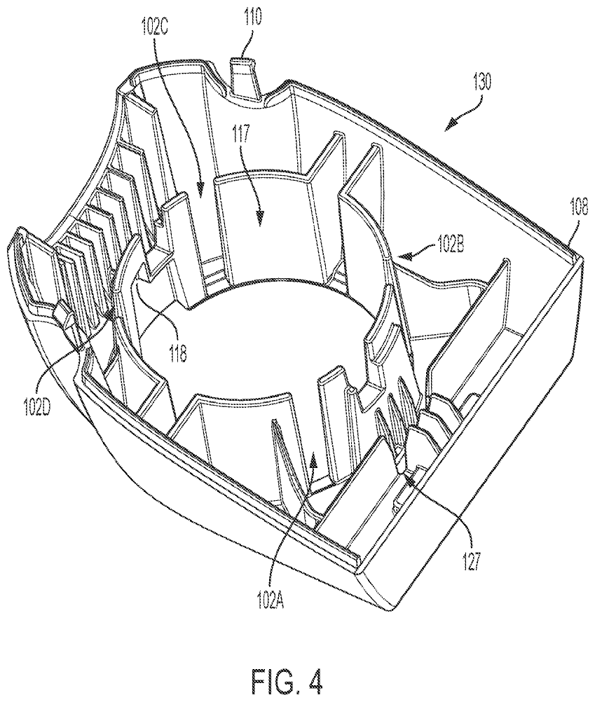

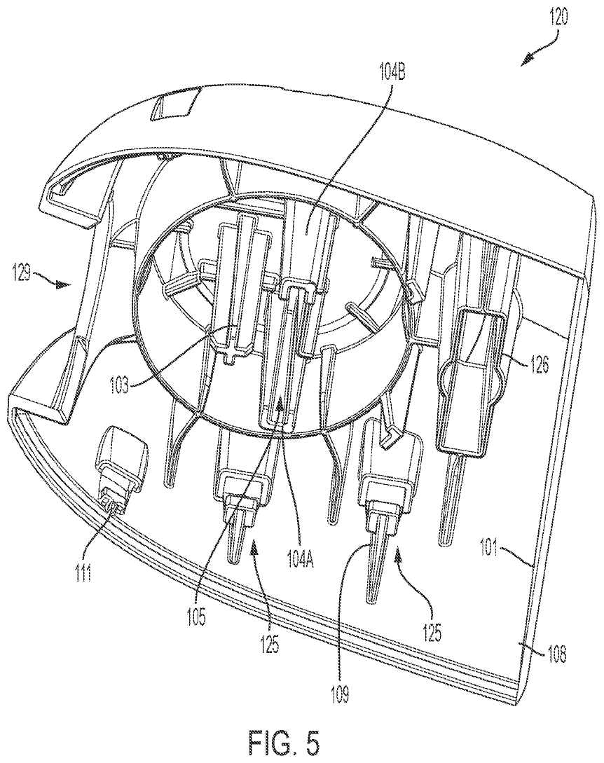

Lower housing 130 is shown in FIG. 4, and upper housing 120 is shown in FIG. 5. Lower housing 130 and upper housing 120 are coupled together along housing interface 108 to hold the other elements of inserter device 10. In the illustrated embodiment, lower housing 130 includes snaps 110 and upper housing 120 includes corresponding latches 111, although this arrangement may vary.

FIG. 4 shows lower housing 130 including an operation channel 117 and a plurality of guide slots, illustratively guide slots 102A, 102B, 102C, 102D. Guide slots 102A, 102B, 102C, 102D are defined by grooves or cutouts in the wall surrounding operation channel 117. Guide slots 102A, 102B, 102C, 102D are spaced apart along the circumference of operation channel 117 so that guide slots 102A and 102B are located towards the rear side of inserter device 10 and guide slots 102C and 102D are located towards the front side of inserter device 10.

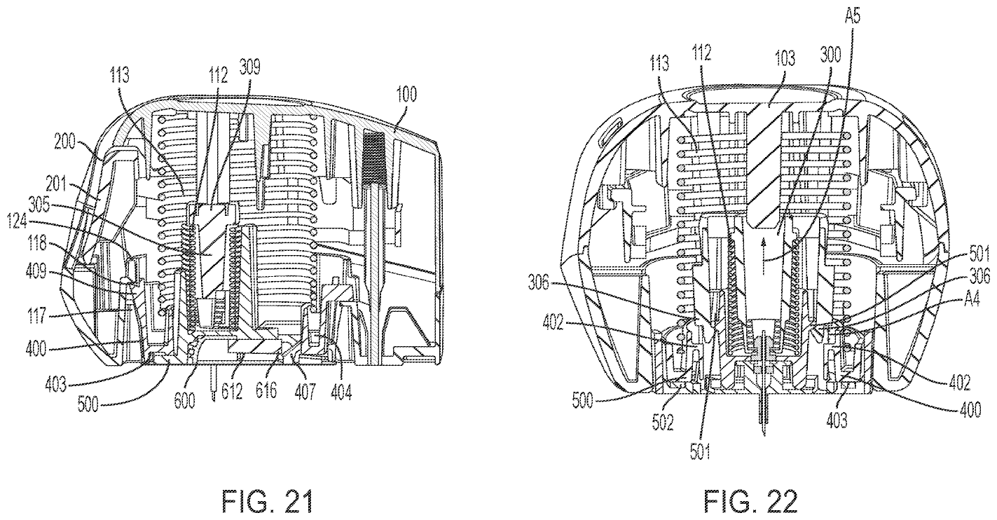

As shown in FIG. 4, lower housing 130 also includes shift plate catch 118. Shift plate catch 118 is disposed near an upper end of operation channel 117. Shift plate catch 118 extends horizontally inward from the wall surrounding operation channel 117 towards the center of operation channel 117. In use, shift plate catch 118 is configured to catch a portion of shift plate 400 and prevent it from retracting after activation of inserter device 10 (FIG. 21). The operation of inserter device 10 will be discussed further below.

Referring still to FIG. 4, lower housing 130 further includes a safety seat 127 configured to receive safety assembly 700 (FIG. 3). Safety seat 127 includes an elongated aperture on the bottom of lower housing 130 that widens as it extends upward into lower housing 130.

FIG. 5 shows upper housing 120 including a central core 103 and guide slots 104A, 104B. Central core 103 extends vertically downward and is configured to guide and encourage only vertically oriented movement of needle assembly 300 (FIG. 3). Guide slots 104A, 104B extend vertically downward and are configured to slidably receive push plate 500 (FIG. 3).

As shown in FIG. 5, upper housing 120 is configured to accommodate carriage 200 (FIG. 3). Upper housing 120 includes button aperture 129 sized and configured to allow button 201 of carriage 200 to extend partially out of upper housing 120 and to be pressed further into button aperture 129. Upper housing 120 also includes a rear carriage return wall 101.

Referring still to FIG. 5, upper housing 120 also includes carriage slots 125. Carriage slots 125 are disposed along the inner walls of upper housing 120. Carriage slots 125 extend horizontally into upper housing 120 and are configured to suspend and slidably retain carriage 200 throughout the pressing and resilient movements involved in the activation of inserter device 10. Carriage slots 125 extend horizontally into upper housing 120 far enough to provide a substantially straight horizontal sliding path for carriage 200 along the length L of inserter device 10 (FIG. 3).

Referring still to FIG. 5, upper housing 120 includes safety column 126 configured to support safety assembly 700 (FIG. 3). Safety column 126 widens as it extends vertically downward from upper housing 120 and cooperates with the above-described safety seat 127 of lower housing 130 (FIG. 4). The operation of safety assembly 700 will be discussed further below.

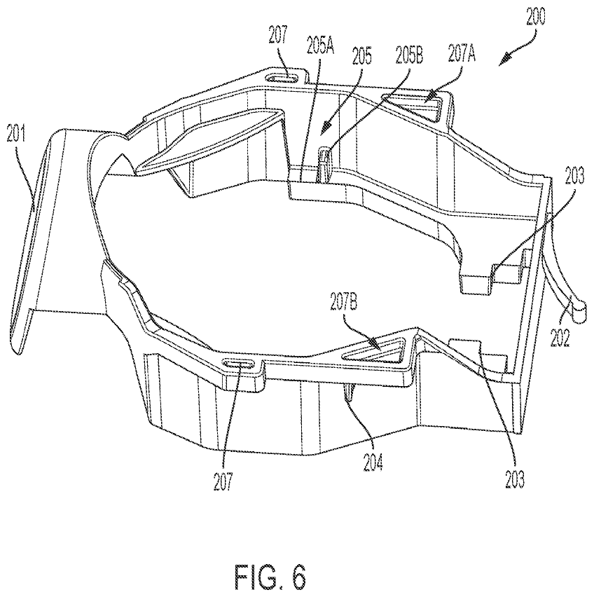

FIG. 6 shows carriage 200 of inserter device 10. Button 201 of carriage 200 extends into button aperture 129 of upper housing 120 (FIG. 5). Button 201 is functionally shaped so that it is ergonomically fitted to the patient's finger or thumb. Carriage 200 may be a molded piece, therefore button 201 is configured to shift the entire carriage 200 upon being pressed into housing 100 (FIG. 3). As shown in FIG. 6, carriage also includes tabs 204. Tab 204 is configured to engage button catch 109 of upper housing 120 (FIG. 5) when button 201 is pressed and shifted into upper housing 120. In this way, tab 204 limits the movement of button 201 so that it is not pushed into upper housing 120 so far as to interfere with other mechanics of inserter device 10. FIG. 6 also shows carriage return spring 202 of carriage 200. Carriage return spring 202 can be a cantilever arm, or any other biasing system known in the art. Carriage return spring 202 is disposed on an opposite side of carriage 200 of button 201 and is configured to engage rear carriage return wall 101 of upper housing 120 (FIG. 5).

As shown in FIG. 6, carriage 200 also includes one or more safety catches 203 and one or more shift plate catches 205 (one of which is hidden from view). FIG. 6 shows that safety catches 203 are stepped flanges that extend inward from carriage 200. FIG. 6 also shows that shift plate catches 205 include a flat portion 205A that extends inward from carriage 200 and forward towards button 201, and vertical portion 205B that also extends inward from carriage 200 and vertically upward.

Referring still to FIG. 6, carriage 200 also includes rails 207A and 207B. Rail 207A and 207B is slidably received within carriage slots 125 of upper housing 120 (FIG. 5). Rails 207A and 207B and carriage slots 125 are configured to suspend carriage 200 within housing 100 so that carriage 200 does not interfere with any other mechanism of inserter device 10 while also allowing carriage 200 to shift horizontally along the length L of housing 100 (FIG. 3) to activate inserter device 10.

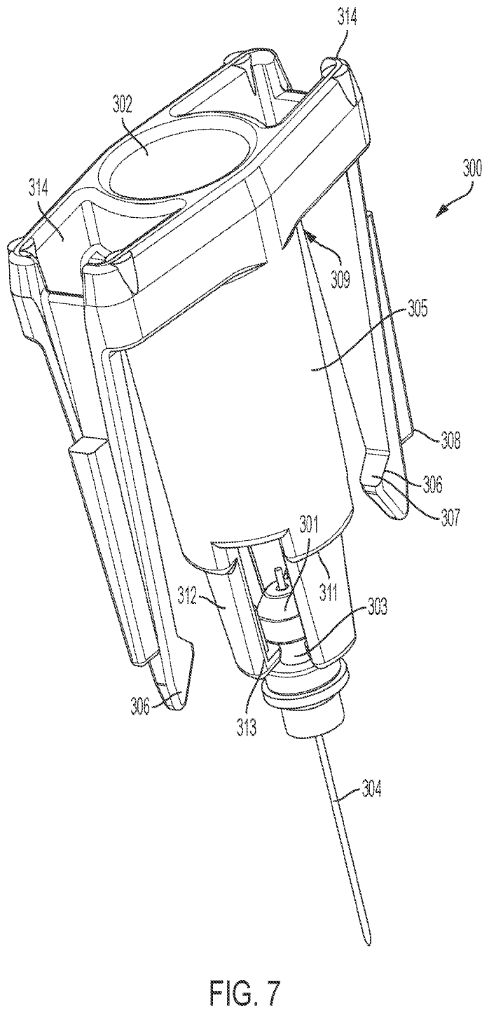

FIG. 7 shows needle assembly 300 of inserter device 10. Needle assembly 300 is comprised of three main components: needle head 301, introducer needle 304 that extends through needle head 301, and needle hub 305 that supports needle head 301. In FIG. 7, needle hub 305 is shown to have circular slot 302, which is substantially uniform in diameter and extends down along the length of needle hub 305. Circular slot 302 is sized and configured to receive central core 103 of upper housing 120 (FIG. 5). At the top of circular slot 302, needle hub 305 also has shoulders 314 that extend radially outward. Extending vertically down from shoulders 314 are snap arms 306. Each snap arm 306 includes an inner push plate lip 307 and an outer shift plate lip 308. Snap arms 306 are configured to be semi-flexible and resilient. As shown in FIG. 7, needle hub 305 includes spring catch 309 disposed on the underside of shoulders 314 around circular slot 302. Spring catch 309 extends radially outward from needle hub 305 and can span the circumference of needle hub 305 or can be discrete extensions spaced around the circumference of needle hub 305. Lastly, needle hub 305 also includes needle head grasps 311. Needle head grasps 311 include arms 312, which extend further downward and are semi flexible and resilient to aid in assembly. At the end of arms 312, are catches 313 which are configured to engage with needle head 301 to hold onto needle head 301 during insertion and retraction.

As shown in FIG. 7, needle head 301 and introducer needle 304 are held horizontally in place by needle head grasps 311. Needle head grasps 311 engage needle head 301 through grasping slot 303. Grasping slot 303 is sized and configured to receive catches 313. The top surface of needle head 301 is angled such that, during assembly, needle head 301 is pushed up into the space between arms 312. As needle head 301 is pushed into arms 312, arms 312 flex outward, then snap catches 313 snap into place into grasping slot 303.

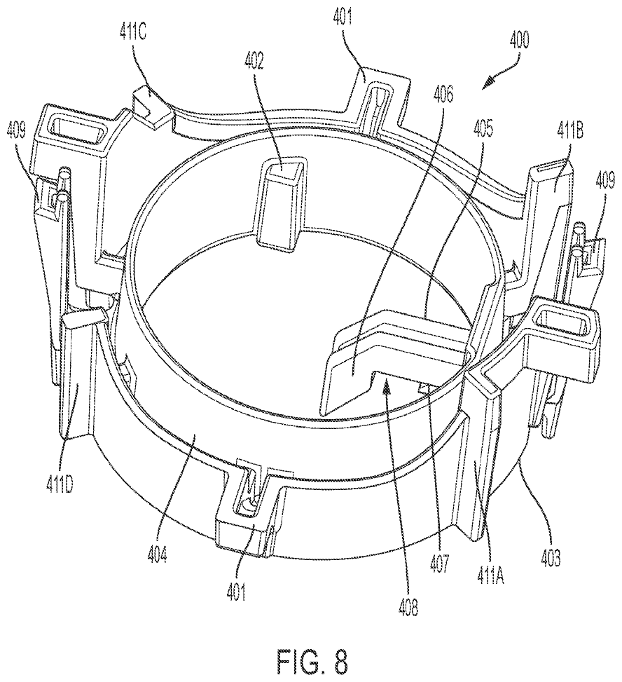

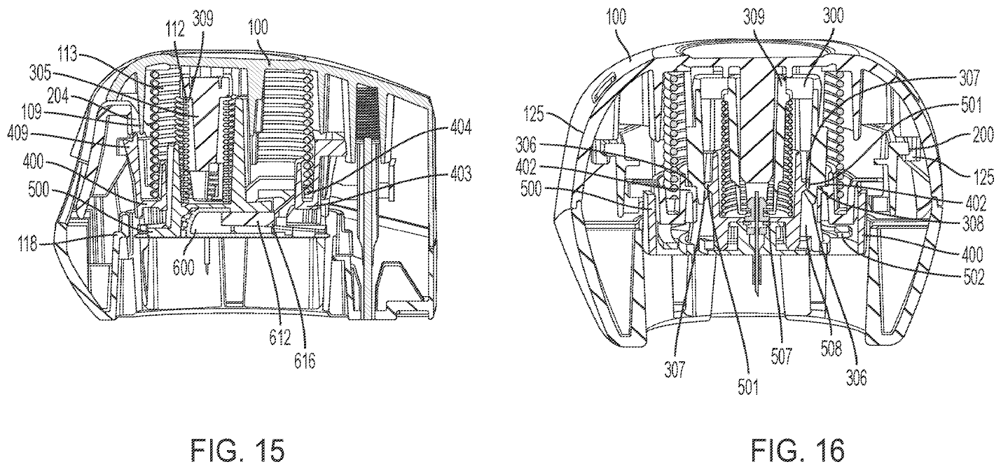

FIG. 8 shows shift plate 400 of inserter device 10. As FIG. 8 depicts, shift plate 400 is a substantially circular component and includes tabs 401 projecting from either side of shift plate 400. Shift plate 400 also includes needle hub catches 402. Needle hub catches 402 extend radially inward from shift plate 400 to form a platform. The bottom of shift plate 400 forms a bottom platform 403 (See also FIG. 15). Bottom platform 403 extends all the way around shift plate 400. Immediately above bottom platform 403 is spring receiving slot 404 which is a U-shaped cavity, the bottom of which is the reverse side of bottom platform 403.

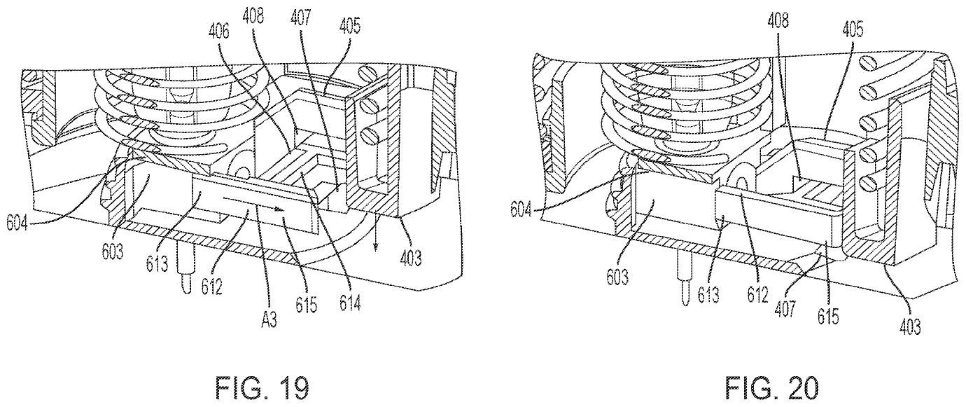

Referring still to FIG. 8, shift plate 400 also includes a headlock engaging arm 405. Headlock engaging arm 405 extends radially inward from shift plate 400 and includes ramped wedge 406 at its innermost end. Underneath headlock engaging arm 405 is ramped receiver 407, which extends radially inward from shift plate 400 below headlock engaging arm 405 (See also FIG. 19), and headlock receiving slot 408. FIG. 8 also shows retention clip 409 of shift plate 400. Retention clip 409 is semi-flexible and configured to be biased inward until shift plate 400 is shifted completely down, at which time retention clip 409 is released to snap under shift plate catch 118 of lower housing 130 (FIG. 4) and lock the position of shift plate 400. To encourage only vertical movement and to discourage lateral or horizontal movements through operation of inserter device 10, shift plate 400 also includes guide rails 411A, 411B, 411C, 411D.

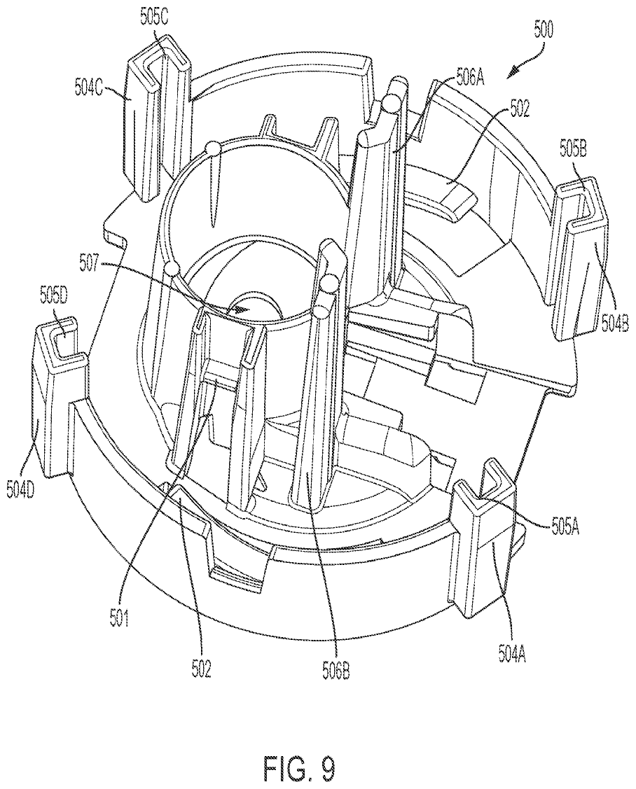

FIG. 9 shows push plate 500 of inserter device 10. Push plate 500 includes needle hub catches 501. Needle hub catches 501 engage snap arms 306 of needle hub 305 (FIG. 7) to pinch snap arms 306 between needle hub catches 501 and needle hub catches 402 (FIG. 8). As shown in FIG. 9, push plate 500 also includes one or more biasing arms 502. Biasing arms 502 are disposed along the bottom surface of push plate 500 and are biased upward but configured to be pressed downward. Biasing arms 502 of the present embodiment are cantilever arms, alternatively, they could be springs, or any other bias known in the art. Referring momentarily to FIG. 16, push plate 500 also includes infusion head cavity 508 and needle head aperture 507. Infusion head cavity 508 is a rounded cavity in the underside of push plate 500 configured to receive infusion head assembly 600. Needle head aperture 507 is an aperture in the top of infusion head cavity 508 which allows needle head 301 to rest atop push plate 500 while needle 304 extend beyond push plate 500.

Returning to FIG. 9, to encourage only vertical movement and to discourage horizontal movement or wobble, push plate 500 also includes outward-facing guide rails 504A, 504B, 504C, 504D which are sized and configured for receipt in corresponding guide slots 102A, 102B, 102C, 102D of lower housing (FIG. 4). Guide rails 504A, 504B, 504C, 504D also include inward-facing guide slots 505A, 505B, 505C, 505D which are sized and configured to receive corresponding guide rails 411A, 411B, 411C, 411D of shift plate 400 (FIG. 8). Push plate 500 further includes guide posts 506A, 506B that extend vertically upward and are slidably received within corresponding guide slots 104A, 104B of upper housing 120 (FIG. 5).

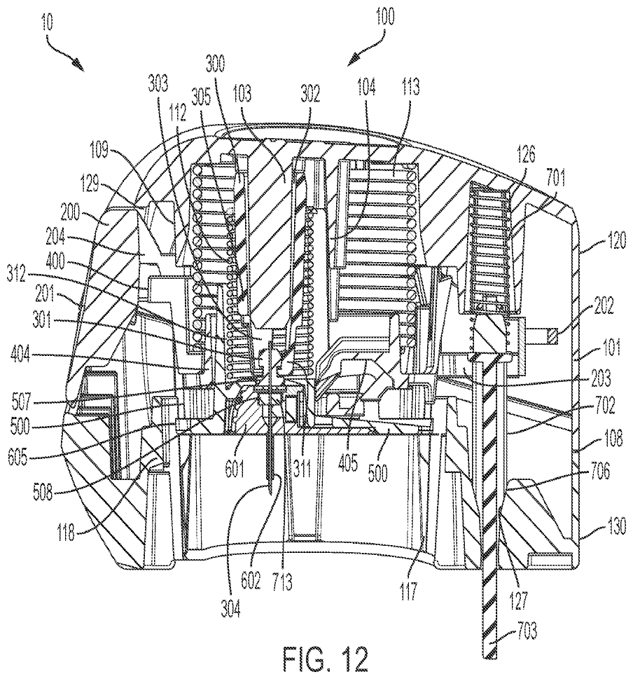

FIG. 12 shows a retraction mechanism in the form of a retraction spring 112 and an automatic insertion mechanism in the form of an insertion spring 113 of inserter device 10. Retraction spring 112 is disposed around needle hub 305 and engages the top surface of infusion head cavity 508 of push plate 500 at its lower end and engages spring catch 309 at its upper end (FIG. 16). Insertion spring 113 is disposed around and within spring receiving slot 404 of shift plate 400 at its lower end and engages the top surface of upper housing 120 at its upper end.

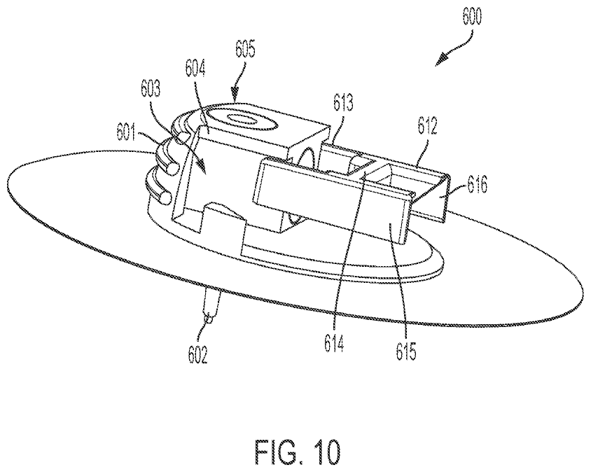

FIG. 10 shows infusion head assembly 600 of inserter device 10. Infusion head assembly 600 is comprised of infusion head 601, cannula 602, and needle cavity 605. Infusion head 601 is the main body of infusion head assembly 600 and is shaped and configured to be received within infusion head cavity 508 of push plate 500 (FIG. 12). Needle cavity 605 is a bore in infusion head 601 that is shaped and configured to receive introducer needle 304 (FIG. 12) to allow introducer needle 304 to extend all the way through infusion head 601 and into cannula 602. Cannula 602 is a soft, thin tube that receives the introducer needle 304 during insertion and that extends into the patient's skin to administer the drug. Infusion head 601 also includes a channel 603 which is sized and configured to slidably receive headlock 612. Channel 603 is defined by lips 604 on at least an upper border of channel 603 which are configured to suspend and mount infusion head assembly 600 to push plate 500 until the insertion operation of inserter device 10 is complete, as described further below.

Referring still to FIG. 10, headlock 612 includes forward-facing infusion headlock arms 613 and rearward-facing push plate engaging arms 615 and a bracket 614 that connects infusion headlock arms 613 and push plate engaging arms 615. Infusion headlock arms 613 extend into channel 603 and engage lips 604; and push plate engaging arms 615 extend into and lock onto push plate 500 (FIG. 9) to lock infusion head assembly, as described further below. Push plate engaging arms 615 include an angled surface 616 to facilitate the engagement of shift plate 400 (FIG. 8) and the unlocking of headlock 612.

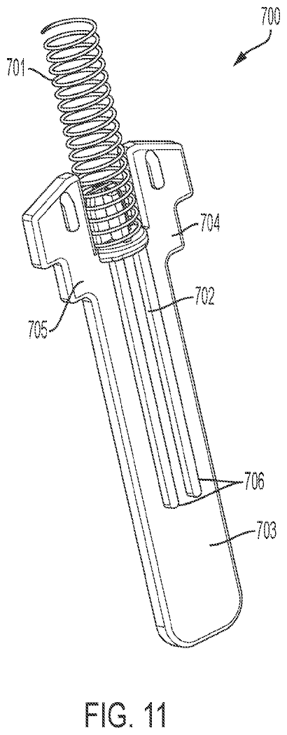

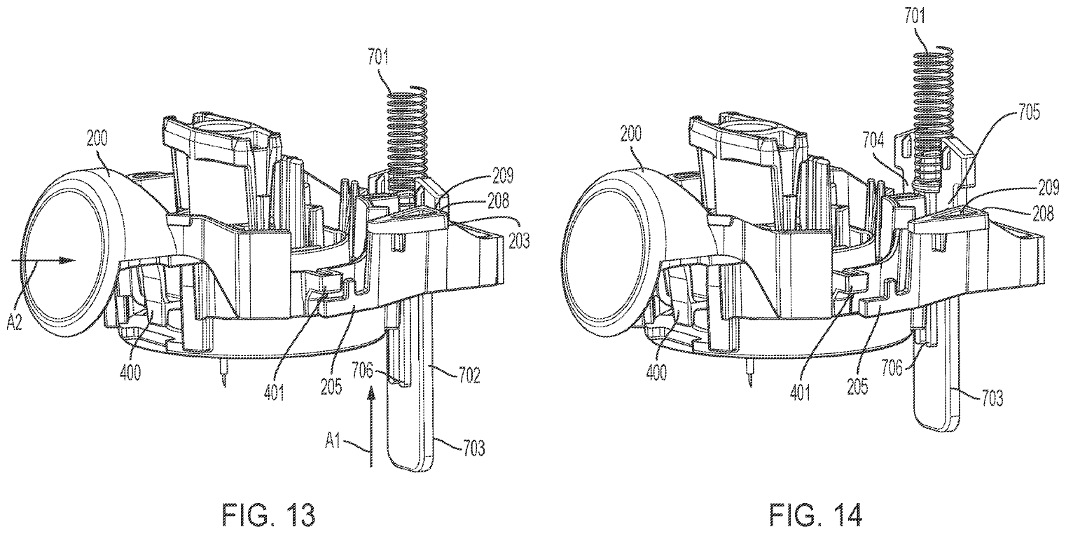

FIG. 11 shows safety assembly 700 of inserter device 10, which is configured to discourage accidental activation of inserter device 10. Safety assembly 700 includes spring 701 which is disposed within safety column 126 of upper housing 120 (FIG. 5) and safety 702 (See also FIGS. 13 and 14). Safety 702 is comprised of a long vertical rod 703 that is partially disposed within safety column 126 of upper housing 120 (FIG. 5) and extends through safety seat 127 in lower housing 130 (FIG. 4). Rod 703 includes tabs 704 near its upper end with side walls 705 that engage safety catch 203 and prevent accidental pressing of button 201 by preventing horizontal movement of carriage 200 (FIG. 6), as described further below. Rod 703 also includes longitudinal ribs 706 that are suspended on and rest on safety seat 127 of housing 100 to retain safety assembly 700 in the housing 100 (FIG. 12).

Operation of inserter device 10 will now be described with reference to FIGS. 12-27.

Initially, as shown in FIGS. 12 and 13, inserter device 10 is provided in a stowed configuration with safety assembly 700 in a locked configuration, in which safety 702 interferes with the translation of carriage 200. The lower end of rod 703 protrudes beneath the housing 100, and the tabs 704 on the upper end of rod 703 engage safety catch 203 of carriage 200 to prevent horizontal movement of carriage 200. Tabs 401 of shift plate 400 are likewise engaged with shift plate catches 205 of carriage 200, holding shift plate 400, push plate 500, needle assembly 300, and infusion head assembly 600 in place and compressing the insertion spring 113. In this stowed configuration, the needle assembly 300 is lifted and concealed within the housing 100 to prevent contact with the patient or the patient's caregiver. Also, carriage return spring 202 is engaged with carriage return wall 101 of upper housing 120 to prevent slack in button 201 due to tolerances and to provide a higher quality of feel to the user.

Referring next to FIG. 14, safety assembly 700 is shown in an unlocked configuration with safety 702 pushed upward in direction A1 (FIG. 13), such as by contacting the patient's skin. This pushing action frees walls 705 of tabs 704 from safety catch 203 so that carriage 200 can then be translated rearward in direction A2 (FIG. 13). FIG. 14 also shows the inserter device 10 in a triggered configuration with carriage 200 translated in direction A2 in response to a pressing of button 201. This triggering action frees tabs 401 of shift plate 400 from their interference with shift plate catches 205 of carriage 200, and allows shift plate 400, push plate 500, needle assembly 300, and infusion head assembly 600 to translate vertically down under the influence of insertion spring 113 (FIG. 15-18). When button 201 of carriage 200 is pressed, carriage return spring 202 is compressed against carriage return wall 101 of the upper housing 120 and the deflection provides a return force to return carriage 200 to its initial position after release of button 201 (not shown).

Referring next to FIGS. 15 and 16, inserter device 10 is still shown in the triggered configuration, just after carriage 200 has translated, but before insertion has begun. Needle hub 305, shift plate 400, and push plate 500 are held together via snap arms 306 on needle hub 305 that are pinched between shift plate 400 and push plate 500, as shown in FIG. 16. The inner-facing push plate lips 307 engage needle hub catch 501 of push plate 500; and the outer-facing shift plate lips 308 engage needle hub catch 402 of shift plate 400. This pinch of needle hub 305 between shift plate 400 and push plate 500 locks these components together and above carriage 200. This locking also prevents needle hub 305 from being retracted before insertion is complete.

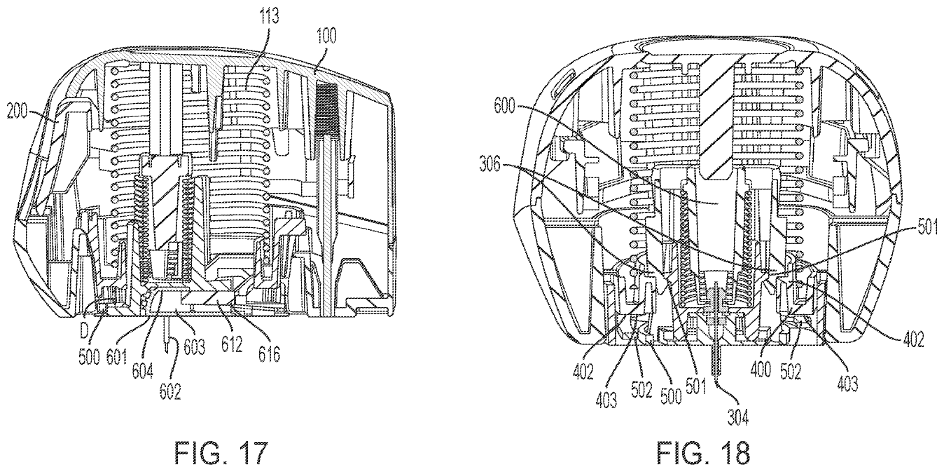

Referring next to FIGS. 17 and 18, inserter device 10 is shown in an inserted configuration. Insertion spring 113 pushes push plate 500, shift plate 400, needle assembly 300, and infusion head assembly 600 downward together past carriage 200. This downward movement continues until push plate 500 bottoms out on the surface to which infusion head 601 is being applied (typically an insertion site on the patient's body). In this inserted configuration, introducer needle 304 and cannula 602 project from housing 100 and into the patient's skin, and infusion head 601 contacts and adheres to the patient's skin. Biasing arms 502 momentarily continue to hold up shift plate 400 by a distance D so that snap arms 306 of needle hub 305 remain pinched between shift plate 400 and push plate 500 and snap arms 306 are thereby prevented from springing outward and releasing needle assembly 300. Push plate 500 may interact with housing 100 to remain locked in the insertion configuration.

Referring next to FIGS. 21 and 22, inserter device 10 is shown in an intermediate configuration. As shift plate 400 continues to translate downwards over distance D (FIG. 17) relative to push plate 500, insertion spring 113 overcomes the force of biasing arms 502 of push plate 500 and forces shift plate 400 to bottom out on push plate 500. During this relative movement, shift plate 400 also continues to move past needle assembly 300, which unlocks snap arms 306 of needle hub 305 so that snap arms 306 are free to deflect upwards and outwards in direction A4, thereby releasing needle assembly 300 from the other components. Retraction spring 112 is then free to drive needle assembly 300 upward in direction A5, as described further below. Shift plate 400 and push plate 500 may be retained in this intermediate configuration based on the continued force from the insertion spring 113 and/or the interaction between retention clips 409 of shift plate 400 and shift plate catch 118 of lower housing 130.

Advantageously, shift plate 400 may trigger retraction of introducer needle 304 based only upon the relative movement of shift plate 400 and push plate 500 and without respect to the ultimate position of push plate 500. For example, even if push plate 500 came to rest on a skin bulge in the insertion configuration without reaching the bottom surface of housing 100, shift plate 400 would continue to move downward and release the introducer needle 304 upon reaching the intermediate configuration. Biasing arms 502 may also serve to dampen the impact of shift plate 400 against push plate 500. The stiffness of biasing arms 502 may be adjusted (e.g. by thickening or thinning the cantilever beam) to control this relative movement between shift plate 400 and push plate 500. Despite these advantages of shift plate 400, it is also within the scope of the present disclosure to eliminate the shift plate 400 in certain embodiments and release the needle assembly 300 based on the position of the push plate 500, for example.

As shown in FIGS. 19 and 20, the shift plate 400 interacts with the headlock 612 to free the infusion head 601 from the inserter device 10. Ramped wedge 406 of shift plate 400 engages with headlock 612 as shift plate 400 is shifted down from the inserted configuration of FIG. 19 to the intermediate configuration of FIG. 20. This engagement pulls headlock 612 rearward towards shift plate 400 in direction A3. Finally, as shift plate 400 bottoms out in the intermediate configuration of FIG. 20, headlock 612 is received within headlock receiving slot 408 which is sized to fit headlock 612. The final, lowered position of ramped wedge 406 holds headlock 612 in the rearward position, preventing free movement of headlock 612.

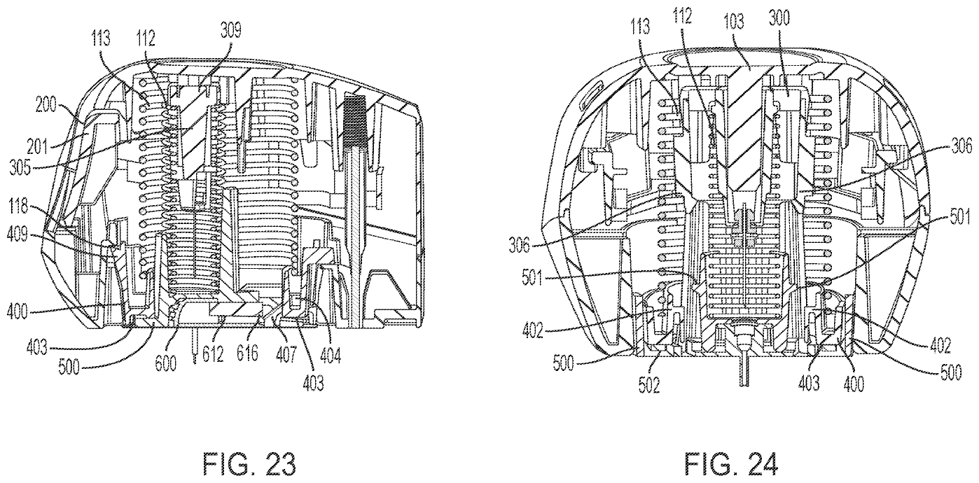

FIGS. 23 and 24 show the inserter device 10 in the retracted configuration once needle hub 305 and introducer needle 304 have retracted into housing 100 by the action of retraction spring 112. In this retracted configuration, inserter device 10 may be lifted comfortably away from the patient's skin. With the infusion head 601 adhered to the patient's skin and the headlock 612 separated from the infusion head 601, as described above, the infusion head 601 and the cannula 602 separate from the inserter device 10 and remain adhered to the patient's skin.

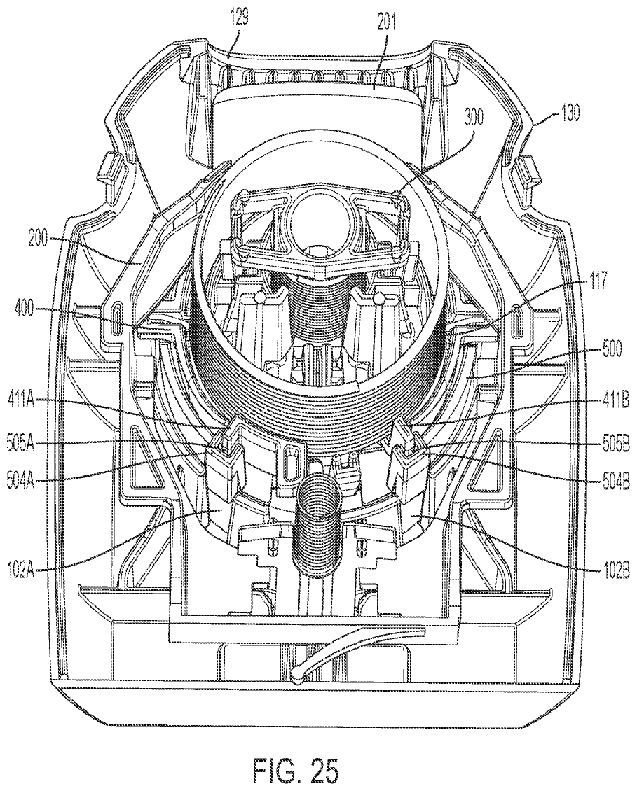

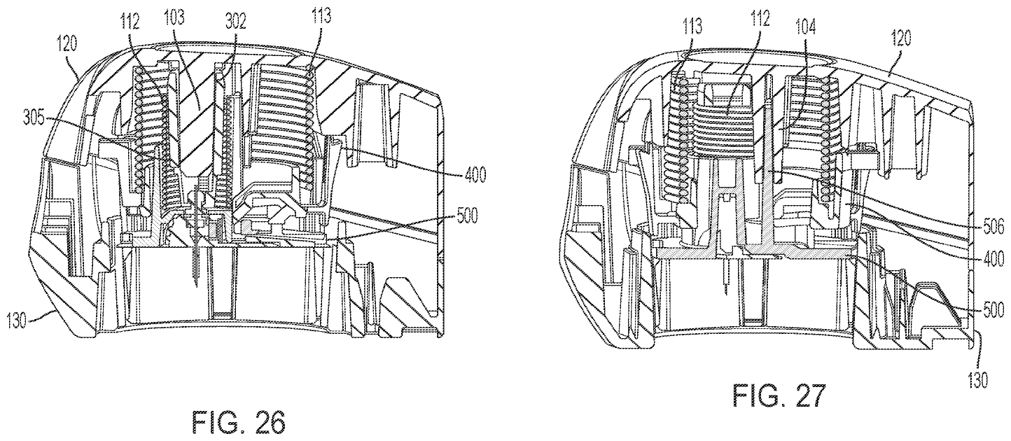

Inserter device 10 may be designed to ensure that introducer needle 304 enters the tissue vertically and perpendicular to the surface of the patient's skin with minimal rotational movement, horizontal movement, or wobble, to ensure patient comfort. Various alignment features are shown in FIG. 25, including guide slots 102A, 102B, 102C, 102D on lower housing 130 (See also FIG. 4) that interact with corresponding guide rails 504A, 504B, 504C, 504D on push plate 500 (See also FIG. 9) and guide slots 505A, 505B, 505C, 505D that interact with corresponding guide rails 411A, 411B, 411C, 411D on shift plate 400 (See also FIG. 8). These alignment features are engaged with one another during the entire vertical translation of infusion head assembly 600. Additional alignment features are shown in FIGS. 26 and 27, including central core 103 of upper housing 120 on which needle hub 305 sits concentrically, and guide slots 104A, 104B of upper housing 120 that interact with guide posts 506A, 506B of push plate 500.

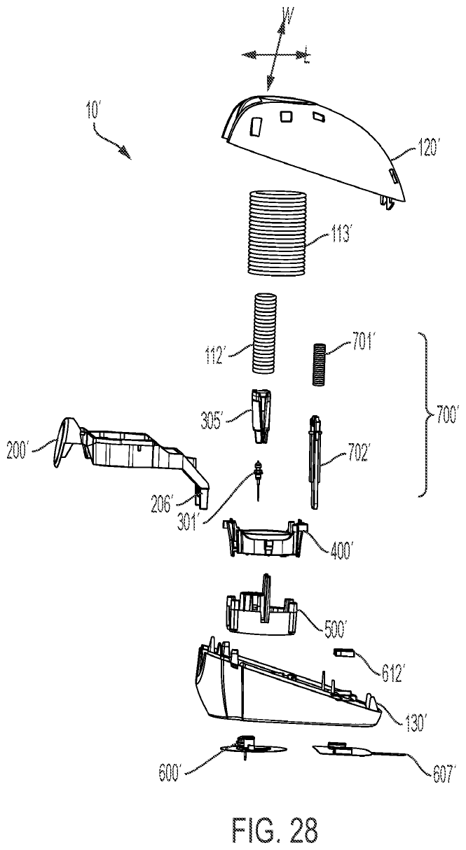

Another exemplary inserter device 10′ is shown in FIGS. 28-31. The inserter device 10′ is similar to the above described inserter device 10, with like reference numerals identifying like elements, except as described below.

Inserter device 10′ is configured to insert introducer needle 304′ and/or cannula 602′ of an infusion head 601′ into the subcutaneous space of the patient, while also enabling application of an optional holder 607′. The holder 607′ may be applied together with the infusion head 601′ or in a subsequent step by aligning the previously-inserted infusion head 601′ with the push plate 500′ to control the position of the holder 607′. Inserter device 10′ is part of a system that enables a drug delivery device such as a pump or bolus injector (not shown) to be worn interchangeably in either a patch modality while coupled to the holder 607′ or a tethered modality without the holder 607′, as described in PCT Application No. PCT/US2020/023825, filed Mar. 20, 2020, the disclosure of which is expressly incorporated herein by reference in its entirety.

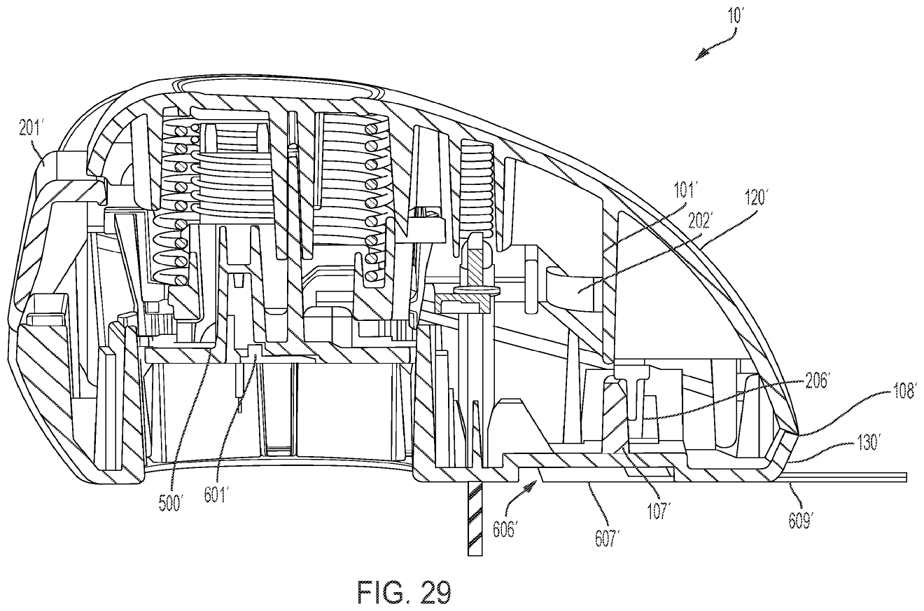

A cross-section of the assembled inserter device 10′ is shown in FIG. 29. Housing 100′ includes a holder receiver 606′ shaped and sized to allow holder 607′ to nest within it. Holder 607′ includes an adhesive backing 609′ configured to adhere to the patient's skin.

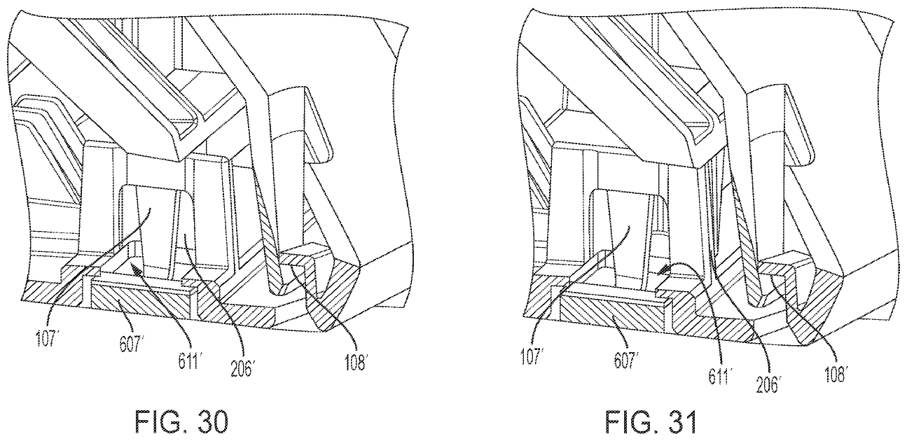

As shown in FIGS. 30 and 31, housing 100′ also includes holder snap 107′. Holder snap 107′ is located towards the rear portion of housing 100′. Holder snap 107′ is a semi-flexible extension of housing 100′ which is reversibly engaged in a snap-fit lock with snap receiver 611′ of holder 607′. In the stowed configuration of FIG. 30, snap blocker 206′ is disposed directly adjacent to holder snap 107′, thus forcing holder snap 107′ into contact with snap receiver 611′ and interfering with movement of holder 607′. Holder snap 107′ is configured to be released from the snap-fit engagement with snap receiver 611′ upon pressing of button 201′. When button 201′ is pressed and carriage 200′ is translated to the triggered configuration of FIG. 31, snap blocker 206′ also translates out of interference with holder snap 107′, thus allowing holder snap 107′ to be released from snap receiver 611′. In preferred embodiments, holder snap 107′ has only a light retaining force, so that the force of adhesive backing 609′ (FIG. 29) of holder 607′ on the skin is enough to pull holder 607′ out from inserter device 10′.

More embodiments and variations of the above disclosure are possible. For instance, the illustrated inserter device 10 uses two compression springs 112, 113 to drive insertion and retraction of introducer needle 304. However, alternative motive forces may be envisioned. For instance, in some embodiments, compression springs 112, 113 may be combined into a double-wound spring. In other embodiments, compression springs 112, 113 are removed, and a scotch yoke, slider crank, torsion spring, or cam-based mechanism might be used to store energy for insertion and retraction of introducer needle 304.

Similarly, the embodiment that has been described thus far uses safety assembly 700, which is released by application of the inserter device 10 to the patient's body; this action thereby pushing safety 702 upward and enabling forward motion of carriage 200. However, safety assembly 700 may take another form; it may be accomplished with a button or slide mechanisms, or by any of various other mechanical mechanisms that are known in the art. In one embodiment, no safety is used.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An inserter device comprising:
a housing;
an actuator;
an infusion head including a cannula;
a push plate removably coupled to the infusion head;
a needle assembly including an introducer needle;
an insertion mechanism configured to automatically move the infusion head, the push plate, and the needle assembly from a stowed configuration in which the cannula and the introducer needle are positioned within the housing to an insertion configuration in which the cannula and the introducer needle extend from the housing in response to movement of the actuator; and
a retraction mechanism configured to automatically move the needle assembly from the insertion configuration to a retracted configuration in which the introducer needle is positioned within the housing.

2. The inserter device of claim 1, wherein the insertion mechanism is an insertion spring and the retraction mechanism is a retraction spring.

3. The inserter device of claim 2, wherein the insertion spring surrounds the retraction spring.

4. The inserter device of claim 2, wherein:
in the stowed configuration, the insertion spring and the retraction spring are both loaded;
in the insertion configuration, the insertion spring is unloaded and the retraction spring is loaded; and
in the retracted configuration, the insertion spring and the retraction spring are both unloaded.

5. The inserter device of claim 1, wherein the retraction mechanism moves the needle assembly to the retracted configuration independent of a location of the push plate relative to the housing.

6. The inserter device of claim 1, further comprising a shift plate, wherein:
in the stowed configuration, the needle assembly is captured between the shift plate and the push plate;
in the insertion configuration, the needle assembly is captured between the shift plate and the push plate; and
in an intermediate configuration between the insertion configuration and the retracted configuration, the shift plate releases the needle assembly from the push plate.

7. The inserter device of claim 6, wherein the push plate includes at least one flexible member that is biased upward in the insertion configuration and is forced downward under the shift plate in the intermediate configuration.

8. The inserter device of claim 6, further comprising a headlock that couples the infusion head to the push plate in the stowed configuration and the insertion configuration and releases the infusion head from the push plate in the intermediate configuration.

9. The inserter device of claim 1, further comprising a headlock that couples the infusion head to the push plate in the stowed configuration and releases the infusion head from the push plate before the retracted configuration.

10. The inserter device of claim 1, further comprising a holder configured for use with the infusion head in a patch modality, wherein the holder is configured to be released from the housing in response to movement of the actuator.

11. An inserter device comprising:
a housing;
an actuator;
an infusion head including a cannula;
a push plate removably coupled to the infusion head;
a needle assembly including an introducer needle;
an automatic insertion mechanism; and
a shift plate having:
a stowed configuration in which the automatic insertion mechanism is loaded against the shift plate with the infusion head and the needle assembly positioned within the housing;
an insertion configuration in which the automatic insertion mechanism moves the shift plate, the infusion head, the push plate, and the needle assembly relative to the housing, the cannula and the introducer needle extending from the housing; and
an intermediate configuration in which the automatic insertion mechanism moves the shift plate relative to the infusion head, the push plate, and the needle assembly.

12. The inserter device of claim 11, further comprising a carriage supporting the actuator, wherein the carriage restrains the shift plate in the stowed configuration and releases the shift plate in the insertion configuration.

13. The inserter device of claim 12, wherein, between the stowed configuration and the insertion configuration, the carriage moves horizontally relative to the housing and the shift plate, the infusion head, the push plate, and the needle assembly move vertically relative to the housing.

14. The inserter device of claim 12, further comprising a safety assembly having a locked configuration that prevents movement of the carriage and an unlocked configuration that permits movement of the carriage.

15. The inserter device of claim 14, wherein the carriage includes a return spring and the housing includes a return wall that engages the return spring to bias the carriage toward the shift plate.

16. The inserter device of claim 15, wherein:
in the locked configuration, the safety assembly extends beneath the housing; and
in the unlocked configuration, the safety assembly is pressed into the housing and above the carriage.

17. The inserter device of claim 11, further comprising a retraction mechanism that automatically moves the needle assembly from the intermediate configuration to a retracted configuration in which the introducer needle is retracted into the housing.

18. The inserter device of claim 17, wherein the automatic insertion mechanism continues to force the shift plate against the push plate in the retracted configuration.

19. The inserter device of claim 17, wherein the shift plate couples to the housing in the intermediate configuration and remains coupled to the housing in the retracted configuration.

20. The inserter device of claim 17, wherein the needle assembly includes at least one snap arm, wherein the shift plate holds the at least one snap arm into engagement with the push plate in the insertion configuration and releases the at least one snap arm from the push plate in the intermediate configuration.

21. The inserter device of claim 11, wherein the push plate is constrained to vertical movement relative to the housing, and the shift plate is constrained to vertical movement relative to the push plate.
